(19) United States
(12) Patent Application Publication
(10) Pub. No.: US 2020/0066398 A1
(43) Pub. Date: Feb. 27, 2020

(54) Nursing Home Bed Reservation

(71) Applicants: Kamal Kejriwal, Redlands, CA (US); Rajesh Bhagaria, Loveland, OH (US)

(72) Inventors: Kamal Kejriwal, Redlands, CA (US); Rajesh Bhagaria, Loveland, OH (US)

(21) Appl. No.: 16/290,232

(22) Filed: Mar. 1, 2019

Related U.S. Application Data
(63) Continuation-in-part of application No. 14/996,157, filed on Jan. 14, 2016, now abandoned.

(51) Int. Cl. G16H 40/20; G16H 10/60; G16H 80/00; A61B 5/00; A61G 12/00; G06Q 50/22

(52) U.S. Cl. CPC G16H 40/20; G16H 10/60; G06Q 50/22; A61B 5/002; A61G 12/00; G16H 80/00

(57) ABSTRACT

A computer software for managing discharge of patients from a hospital to a nursing care facility has the steps of a nursing facility registers on the database, the nursing facility providing real-time updates on a current bed availability, the database is updated with the bed availability as well as features of the available bed, an up-to-date view of bed availability is provided to a hospital case managers view bed availability, and the patient is discharged and sent to the available bed. The features are selected from the group consisting of gender, isolation beds, subacute conditions, custodial beds and the presence of a ventilator.

### CROSS-REFERENCE TO RELATED APPLICATION

The present application is a Continuation-in-Part Application of U.S. Non-Provisional patent application Ser. No. 14/996,157 filed on Jan. 14, 2016 titled “Nursing Home Bed Reservation System” which was in turn a Continuation in-Part application of U.S. Non-Provisional patent application Ser. No. 14/108,172 filed on Feb. 14, 2014, entitled “Improving Patient Transition by Web Based program to have real time availability of Skilled nursing home beds.”, the entire disclosure of which is incorporated by reference herein.

### BACKGROUND OF THE INVENTION

### 1. Field of Invention

The present invention relates to the field of software used to facilitate nursing home bed reservations and transfers.

### 2. Description of Related Art

The current mechanism for discharging a patient from a hospital to a nursing home or other alternative care facility often delays patient discharge, resulting in unnecessarily prolonged occupancy of hospital beds. Currently, when the patient is ready to be discharged from the hospital, the hospital's discharge planners call the local network nursing care facilities to determine both the specific care needs the facilities are equipped to provide and their respective bed availability. In addition to being an unduly burdensome and time-consuming process, if the discharge planners attempt to contact the nursing care facilities after hours or on a weekend, they are unable to reach the appropriate staff to determine the facilities' bed availability. Only after bed availability has been determined and a decision on facility choice has been made, will the patient be discharged to the respective facility. This unnecessarily ties up hospital beds and other resources, as the hospital administrators wait for notification that a bed is available. This also affects the nursing home revenues as their bed may potentially go infilled due to current system.

Based on the foregoing, there is a need in the art for means that enables hospitals to check real-time availability of care facility beds, thus eliminating unnecessary delay of patient discharge from the hospital to the care facility.

### SUMMARY OF THE INVENTION

In a mechanism for managing discharge of patients from a hospital to an alternative care facility, the nursing care facilities register on a website and provide real-time updates of their current bed availability. The hospital case managers and discharge planners have access to the website, allowing them to view real time bed availability at the registered facilities at any given time. This allows patients who are ready for, or are anticipating, discharge from the hospital to be discharged to an appropriate care facility in an efficient manner, thus increasing hospital bed availability for incoming patients. Furthermore, in the event the patient requires skilled care, the patient is able to transition to the care facility and begin skilled nursing treatment sooner.

A computer program for managing discharge of patients from a hospital to a nursing care facility has the steps of a nursing facility registers on the database, the nursing facility providing real-time updates on a current bed availability, the database is updated with the bed availability as well as features of the available bed, an up-to-date view of bed availability is provided to a hospital case managers view bed availability, and the patient is discharged and sent to the available bed.

The features are selected from the group consisting of gender, isolation beds, subacute conditions, custodial beds and the presence of a ventilator. The system may also have the step of placing a biomonitoring device on a patient and monitoring the patient. Monitoring the patient may include locating the patient within the facility, monitoring vital functions, and monitoring for early stroke detection.

The vital functions monitored are selected from the group consisting of pulse rate, respiration rate, blood oxygen levels, blood glucose levels, and tremors. The system may have early stroke detection that includes monitoring a baseline and notifying the patient if the patient vitals move away from the baseline.

Locating the patient within the facility is achieved by transmitters worn by patients providing location of the patient with reference to anchor nodes. In an embodiment, the patient may select a particular location within the facility based on a floorplan of the facility. If multiple facilities have available beds then the patient or the patients' family selects the facility from the multiple facilities. To help with the decision, additional features like videos and pictures of nursing care facilities, beds and instant chatting with Nursing Home staff will be available.

The foregoing, and other features and advantages of the invention, will be apparent from the following, more particular description of the preferred embodiments of the invention, the accompanying drawings, and the claims.

### BRIEF DESCRIPTION OF THE DRAWINGS

For a more complete understanding of the present invention, the objects and advantages thereof, reference is now made to the ensuing descriptions taken in connection with the accompanying drawings briefly described as follows.

FIG. 1 is a flowchart of the method, according to an embodiment of the present invention; and

FIG. 2 is a functional diagram of the system, according to an embodiment of the present invention.

### DETAILED DESCRIPTION OF PREFERRED EMBODIMENTS

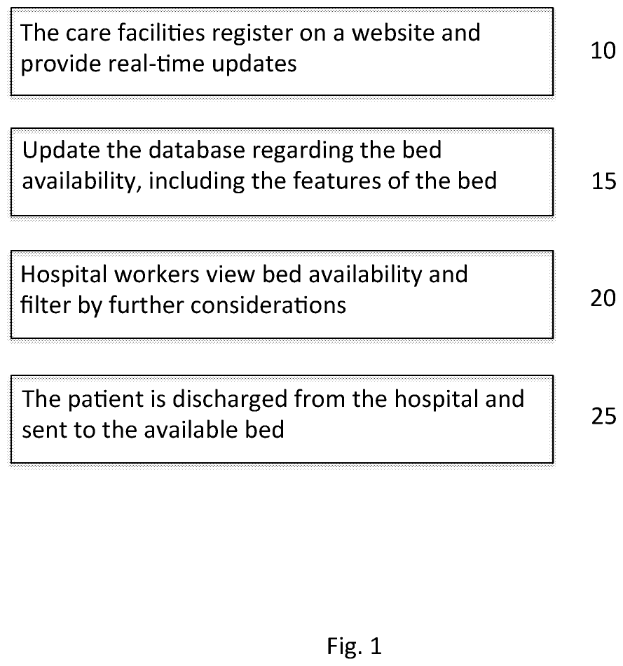

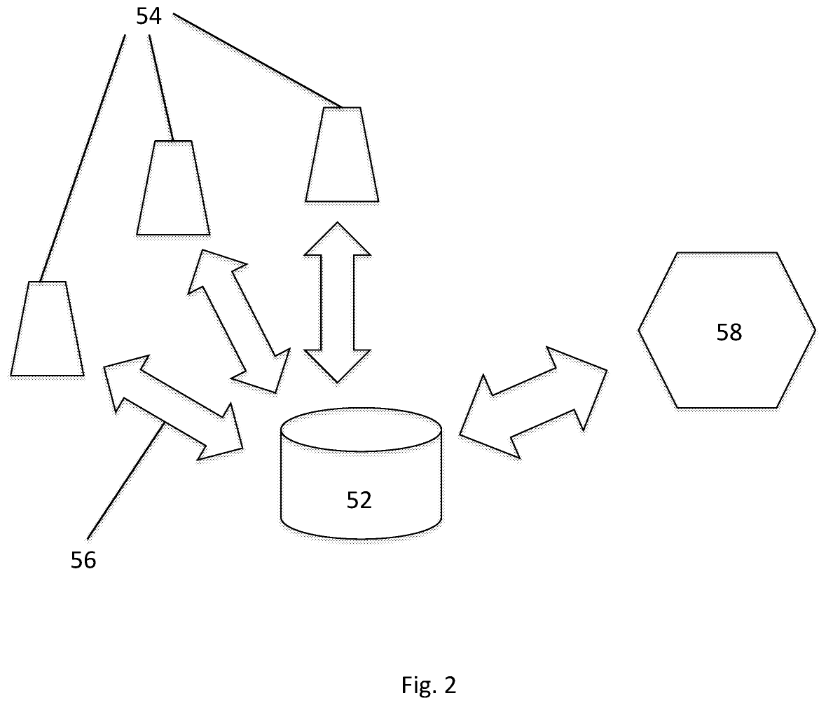

Embodiments of the present invention and their advantages may be understood by referring to FIGS. 1-2, wherein like reference numerals refer to like elements.

With reference to FIG. 1, and in a system for managing discharge of patients from a hospital to an alternative care facility, in step 10 the nursing care facilities register on a website and provide real-time updates of their current bed availability. In an embodiment, the connections from the nursing care facilities to the database are secure tunnels or VPNs.

The website is connected to a database at its back end and in step 15 updates the database regarding the bed availability, including the features of the bed available, including whether the beds are for males or females, if they are isolation beds for certain conditions, the total capacity of facility to ensure every bed is accounted for, subacute conditions that may be treated in the bed, the presence of long-term custodial beds and the presence of a ventilator.

The data is updated in real time to provide an up-to-date view of the bed availability in various nursing care facilities, and may be updated automatically by interfacing with the nursing home database.

In an embodiment the data on patients is received by means of an electronic biomonitoring device that fits on or implanted within the patient's body and monitors presence (through an indoor GPS) as well as vital functions such as pulse rate, respiration rate and oxygen levels, temperature, blood pressure, blood glucose levels, tremors early stroke or seizure detection. The early stroke detection monitors the body for a baseline and shocks the body if the monitored vitals move outside of the normal range, potentially indicating a stroke or heart attack. The collected data is sent data wirelessly to the database.

The indoor GPS or indoor positioning system may operate by RFID, Bluetooth or Wi-Fi transmitters on the patients and anchor nodes at known locations within the indoor space.

In step 20 the hospital case managers and discharge planners have access to the website, allowing them to view bed availability at the registered facilities at any given time, with the ability to sort by patient gender, and whether an isolation bed, subacute conditions treatment or a ventilator is required, for example. This allows patients who are ready for, or are anticipating, discharge from the hospital to be discharged to an appropriate care facility in an efficient manner avoiding delays, thus increasing hospital bed availability for incoming patients. If only one bed is available, the patient has no choice and is assigned to that bed, with the purpose of clearing up bed space in the hospital. This is particularly useful for freeing up beds on the weekend, as in the prior art manual process, beds are only reported as empty during the week. If beds available in more than one alternative care facility that meet the patient's needs, the patient or patient's family are given options on which nursing home to go to. To help with the decision, additional features like videos and pictures of nursing care facilities, beds and instant chatting with Nursing Home staff will be available. If they don't make a choice, and remain in the hospital, further charges will be incurred by the hospital.

Once a bed is identified, at step 25 the patient is discharged from the hospital and sent to the available bed. Furthermore, in the event the patient requires skilled care not available at the hospital, the patient is able to transition to the care facility and begin skilled nursing treatment sooner. The patient's bed may be located by maps within an overview of the facility floorplan, or using indoor GPS, and the patient of patient's family may select a particular location within the nursing care facility for the patient. Some patients would prefer to be closer to the nurses' station, while others may want to be out of the way in a corner.

With reference to FIG. 2, the database is shown with connections from all the nursing care facilities. Further, secure hospital connections with the database permit the hospital to see bed availability in real time communication, instantaneously monitoring bed availability in a network of nursing homes.

The criteria that are considered in a decision on whether available beds are, for example, patient gender, and whether an isolation bed, subacute conditions treatment or a ventilator is required. Other possible considerations are long term stay beds.

With reference to FIG. 2, once the decision is made to discharge the patient from the hospital, the discharge planners will go on the created website where they wilt have a real time updates of the nursing home bed availability in the geographical area. They can secure that bed on the secure website, which will help in early and safe transition of patient from hospital to nursing home. The database 52 contains the data records on available beds in real-time. The nursing facilities 54 are connected to the database securely via encrypted connections 56 such as VPN or secure tunnels. Data is provided by the facilities as soon as the bed situation changes, so as to maintain the database in an up-to-date form. The hospital 58 queries the database according to the criteria when a patient is ready for discharge to a bed, and the database returns the location of a bed through a secure connection.

The invention has been described herein using specific embodiments for the purposes of illustration only. It will be readily apparent to one of ordinary skill in the art, however, that the principles of the invention can be embodied in other ways. Therefore, the invention should not be regarded as being limited in scope to the specific embodiments disclosed herein, but instead as being fully commensurate in scope with the following claims.

In the preferred embodiment, a staff at the admitting hospital may begin a repatriation event using a graphical user interface though use of a computer terminal or via a mobile device such as a smart phone or a smart tablet. For example, a staff or a case manager may log in using a web portal by entering user name and a secure password. Any user must register and be approved before being able to log in through the web portal.

Each nursing care facility has a profile page which displays relevant information about the facility such as address, contact information, mailing address, website, pictures, videos and alike. All nursing care facilities must be registered with the web portal prior to their information being accessible by users. Each nursing care facility's available beds are displayed on a separate page including classifications of each bed such as male, female, isolated, custodial (long term care) etc.

A user can check availability of beds based on different criteria e.g. zip code, city, county etc. A user can also access feature of each facility such as pictures, videos, ratings, and other relevant information about each facility. The user can use an instant chat box to communicate with a representative from nursing care facilities regarding any questions the patient or his/her family might have about the facility. Star ratings regarding health inspection, staffing and quality of care of the nursing home from Medicare.gov will be available to hospital staff/Patient/Care taker so that they can take a more informed decision. In addition, nursing home compare based on star ratings will be available. There will be a link for accommodation options as well as recreational activities near the nursing care facilities: hotels and recreational choices in nearby areas will be made visible. These will be visible to the Patient/Care taker so that they can take a more informed decision. A user can request a bed or a room by pressing “Hold” button. The user can also send and receive test messages along with room request to inform or inquire about certain relevant information. The nursing care facility can accept or decline a room request based on their capacity and other relevant data. Once the relevant input has been entered by the case manager. The software automatically matches the patient with nursing care facilities which fulfill the selected criteria.

1. A non-transitory storage medium having stored thereon a computer program for repatriating a patient from a hospital to a nursing care facility, said program comprising:
first instructions that when executed by a computer hardware, automatically matches the patient to one or more of accepting nursing care facilities, wherein the first instructions cause said computer hardware to access from said storage medium patient data and participating nursing care facilities data and automatically matching said patient to one or more of the said nursing care facilities based on pre-determined criteria; said predetermined criteria consisting of gender, isolation status, subacute condition, custodial, presence of a ventilator, availability of lodging in the area, recreational facilities in the surrounding area, rating of nursing care facilities, video and pictures of nursing care facilities, instant chatting, insurance provider and ratio of residents per staff;
second instructions that when executed by computer hardware handle registration and selection of nursing home care facilities and repatriation enquiry, wherein second instructions cause the computer hardware to automatically generate a list of nursing care facilities that match patient's needs, electronically display said list, electronically receive a repatriation request from admitting hospital, electronically transmit said request to selected nursing care facility, and electronically receive an acceptance of said repatriation request from said selected nursing care facility, and electronically transmit acceptance to the admitting hospital.

2. A method for repatriating a patient from an admitting hospital to a nursing care facility, the method comprising:
automatically matching said patient to one or more nursing care facilities, wherein said matching comprises retrieving from a computer storage medium said patient's data consisting of gender, isolation status, subacute condition, custodial, presence of a ventilator, availability of lodging in the area, recreational facilities in the surrounding area, rating of nursing care facilities, insurance provider and ratio of resident per staff; retrieving from a computer storage medium data regarding candidate nursing care facilities, and automatically matching using computer hardware and software said patient to one or more said nursing home facilities; processing via computer hardware and software a request to repatriate said patient from said admitting hospital to said nursing care facility wherein processing comprises: electronically displaying to the admitting hospital a list of one or more candidate nursing care facilities matched to said patient, electronically receiving a repatriating request from said admitting hospital, said request indicating a selected nursing care facility from one or more candidate nursing care facilities matched to said patient, electronically transmitting said request to said selected nursing care facility, electronically receiving and displaying an acceptance of said repatriation request to the admitting hospital.
